(19) United States
(12) Patent Application Publication
(10) Pub. No.: US 2022/0196596 A1
(43) Pub. Date: Jun. 23, 2022

(54) DEVICES, METHODS, AND SYSTEMS FOR MANIPULATING PROTEINS IN BIOELECTRONIC CIRCUITS

(71) Applicant: RECOGNITION ANALYTIX, INC., Phoenix, AZ (US)

(72) Inventor: Jacob Swett, PHOENIX, AZ (US)

(73) Assignee: RECOGNITION ANALYTIX, INC., PHOENIX, AZ (US)

(21) Appl. No.: 17/559,529

(22) Filed: Dec. 22, 2021

Related U.S. Application Data
(60) Provisional application No. 63/128,978, filed on Dec. 22, 2020.

(51) Int. Cl. G01N 27/447; B03C 5/00

(52) U.S. Cl. CPC G01N 27/44713; G01N 27/4473; B03C 5/005

(57) ABSTRACT

The present disclosure provides devices, systems, and methods related to protein bioelectronics. In particular, the present disclosure provides devices, systems, and methods for manipulating a protein-of-interest into a target position within two electrodes in order to generate a functional bioelectronic circuit. The present disclosure also provides devices, systems, and methods for selectively attracting and concentrating one or more target analytes to the protein-of-interest, which can be used to develop analytical platforms to detect and measure various characteristics of protein function.

### RELATED APPLICATIONS

This application claims priority to and the benefit of U.S. Provisional Patent Application No. 63/128,978 filed Dec. 22, 2020, which is incorporated herein by reference in its entirety for all purposes.

### FIELD

The present disclosure provides devices, systems, and methods related to protein bioelectronics. In particular, the present disclosure provides devices, systems, and methods for manipulating a protein-of-interest into a target position within two electrodes in order to generate a functional bioelectronic circuit. The present disclosure also provides devices, systems, and methods for selectively attracting and concentrating one or more target analytes to the protein-of-interest, which can be used to develop analytical platforms to detect and measure various characteristics of protein function and detect, characterize, and quantify target analytes.

### BACKGROUND

As proteins perform their various functions, movements are generated that underlie these functions. Furthermore, as proteins interact with other molecules, fluctuations may be generated that are characteristic of the interaction. The ability to develop devices, systems, and methods that measure the electrical characteristics corresponding to the fluctuations generated by an protein can be a basis for label-free detection and analysis of protein function. For example, monitoring the functional fluctuations of an active enzyme may provide a rapid and simple method of screening candidate drug molecules that affect the enzyme's function. In other cases, the ability to monitor the fluctuations of proteins that process biopolymers (e.g., carbohydrates, polypeptides, nucleic acids, and the like), or to monitor the fluctuations of proteins as they interact with their target analytes may reveal new information about their conformational changes and how those changes are linked to function. To facilitate this, there is a need to develop bioelectronic platforms and corresponding methods that can efficiently and effectively manipulate the positioning of a protein-of-interest to establish a bioelectronic circuit, which can serve as a basis for the development of diagnostic and analytical devices that take advantage of the electrical characteristics produced by active proteins, providing new ways to leverage biomechanical properties for practical use.

### SUMMARY

Embodiments of the present disclosure include a method for generating a bioelectronic circuit. In accordance with these embodiments, the method includes generating an electric field gradient between an electrode pair functionalized with recognition molecules; exposing the electrode pair to a solution comprising a plurality of proteins-of-interest, wherein each of the plurality of proteins-of-interest comprises two binding sites for interacting with the recognition molecules on the electrode pair; and applying a pre-determined AC voltage and frequency to the electrode pair and attracting a protein-of-interest to the recognition molecules on the electrode pair. In some embodiments, binding of a single protein-of-interest to the electrode pair generates a functional bioelectronic circuit.

In some embodiments, the electric field gradient is generated by applying an initial AC voltage and an initial DC voltage across an electrode pair.

In some embodiments, the pre-determined AC voltage and frequency applied result in dielectrophoresis, thereby attracting the single protein-of-interest to the electrode pair and facilitating binding of the recognition molecules to the binding sites of the protein-of-interest.

In some embodiments, the binding of the single protein-of-interest causes an increase in current from about 1-10 pA to about 100-1000 pA across the circuit. In some embodiments, the binding of the single protein-of-interest causes a decrease in impedance across the circuit. In some embodiments the binding of the single protein-of-interest causes a characteristic change in the conductance fluctuations.

In some embodiments, the method further comprises reducing the pre-determined AC voltage applied to the electrode pair upon the increase in current or decrease in impedance.

In some embodiments, reducing the pre-determined AC voltage applied to the electrode pair upon the increase in current or decrease in impedance stops the attraction of a second protein-of-interest to the electrode pair.

In some embodiments, the method further comprises adjusting the pre-determined AC frequency applied to the electrode pair upon the increase in current or decrease in impedance. In some embodiments, adjusting the pre-determined AC frequency applied to the electrode pair upon the increase in current or decrease in impedance repels a second protein-of-interest from the electrode pair.

In some embodiments, each electrode in the electrode pair is separated by a gap from about 1 nm to about 10 nm.

In some embodiments, the initial DC voltage is from about 5 mV to about 500 mV.

In some embodiments, the pre-determined AC frequency is from about 1 kHz to about 50 MHz.

In some embodiments, the method further comprises exposing the electrode pair to at least a second plurality of proteins-of-interest, and applying a second pre-determined AC voltage and frequency corresponding to the second plurality of proteins-of-interest to the electrode pair.

In some embodiments, the method further comprises exposing a second electrode pair to a second solution comprising a second plurality of proteins-of-interest, and applying a second pre-determined AC voltage and frequency corresponding to the second plurality of proteins-of-interest to the second electrode pair.

In some embodiments, the protein-of-interest is selected from the group consisting of an enzyme, a cell surface receptor, a transmembrane protein, an antibody, an intracellular signaling protein, a growth factor, a nucleic acid binding protein, a secretory protein, viral structural proteins, membrane fusion protein, and any fragments, derivatives, or variants thereof. In some embodiments, the protein-of-interest is selected from the group consisting of a polymerase, a nuclease, a proteasome, a glycopeptidase, a glycosidase, a kinase and an endonuclease.

In some embodiments, the recognition molecules are selected from the group consisting of antibodies, antigen, receptors, and ligands.

In some embodiments, the protein-of-interest is coupled to a carrier (e.g., a gold nanoparticle).

Embodiments of the present disclosure also include a method for increasing concentration of an analyte at a bioelectronic circuit. In accordance with these embodiments, the method includes exposing a bioelectronic circuit to a solution comprising a plurality of analytes, the bioelectronic circuit comprising an electrode pair bound to a protein-of-interest; and generating an electric field gradient between the electrode pair to polarize the plurality of analytes, thereby forcing the analytes to reach an electric field maximum.

In some embodiments, the electric field gradient is generated by applying an initial AC voltage and an initial DC voltage across an electrode pair. In some embodiments, the protein-of-interest is selected from the group consisting of an enzyme, a cell surface receptor, a transmembrane protein, an antibody, an intracellular signaling protein, a nucleic acid binding protein, a secretory protein, and any fragments, derivatives, or variants thereof. In some embodiments, the protein-of-interest is selected from the group consisting of a polymerase, a nuclease, a proteasome, a glycopeptidase, a glycosidase, a kinase and an endonuclease. In some embodiments, the plurality of analytes is a biopolymer or a subunit of a biopolymer.

Embodiments of the present disclosure also include a method for decreasing concentration of an analyte at a bioelectronic circuit. In accordance with these embodiments, the method includes exposing a bioelectronic circuit to a solution comprising a plurality of analytes, the bioelectronic circuit comprising an electrode pair bound to a protein-of-interest; and generating an electric field gradient between the electrode pair to polarize the plurality of analytes, thereby forcing the analytes to reach an electric field minimum.

In some embodiments, the electric field gradient is generated by applying an initial AC voltage and an initial DC voltage across an electrode pair. In some embodiments, the protein-of-interest is selected from the group consisting of an enzyme, a cell surface receptor, a transmembrane protein, an antibody, an intracellular signaling protein, a nucleic acid binding protein, a secretory protein, and any fragments, derivatives, or variants thereof. In some embodiments, the plurality of analytes is a biopolymer or a subunit of a biopolymer.

### BRIEF DESCRIPTION OF THE DRAWINGS

FIG. 1: Representative schematic diagram illustrating a junction for positioning a protein-of-interest in a target region with an AC bias applied, according to one embodiment of the present disclosure.

FIG. 2: Representative schematic diagram of the device of FIG. 1 exposed to a solution comprising proteins-of-interest, each comprising two sites for interacting with a pair of electrodes in the device, according to one embodiment of the present disclosure.

FIG. 3: Representative schematic diagram illustrating a bioelectronic circuit in which a single protein molecule serves to detect target analyte molecules (labeled “T”) in solution, according to one embodiment of the present disclosure.

FIG. 4: Representative schematic diagram illustrating a circuit for actively manipulating the positioning of a protein-of-interest to establish a bioelectronic circuit, according to one embodiment of the present disclosure.

FIG. 5: Representative schematic diagram illustrating methods involving the application of a local AC field for localized Joule heating, according to one embodiment of the present disclosure.

FIG. 6: Representative schematic diagram illustrating methods for selective functionalization of an array of electrode gaps, according to one embodiment of the present disclosure.

FIGS. 7A-7C: Representative data obtained from three separate bioelectronic devices demonstrating increased conductance readings (red trace) after DEP in 1 nM AuNPs after 5 mins under +/−800 mV (FIG. 7A), in 1 nM AuNPs after 10 mins under +/−800 mV (FIG. 7B), and in 1 nM AuNPs after 5 mins under +/−1V (FIG. 7C).

FIGS. 8A-8B: Representative data from controls showing no conductance before the addition of water and AuNPs (FIG. 8A) and in water before addition of AuNPs (FIG. 8B).

### DETAILED DESCRIPTION

Embodiments of the present disclosure provides devices, systems, and methods related to protein bioelectronics. In particular, the present disclosure provides devices, systems, and methods for manipulating a protein-of-interest into a target position within two electrodes in order to generate a functional bioelectronic circuit. The present disclosure also provides devices, systems, and methods for selectively attracting and concentrating one or more target analytes to the protein-of-interest, which can be used to develop analytical platforms to detect and measure various characteristics of protein function.

In accordance with these embodiments, the present disclosure provides devices comprising a first electrode and a second electrode, each electrode functionalized so as to manipulate a protein-of-interest to a target region, and corresponding methods for attracting, concentrating, and detecting the captured protein-of-interest, thereby completing an electrical circuit between the electrodes. In some embodiments, the devices and systems of the present disclosure are configured to enable the active positioning of not more than one protein-of-interest to a gap within a pair of electrodes. Additionally, methods for concentrating a target analyte that can interact with the protein-of-interest at each pair of electrodes are also described herein.

In some embodiments a first electrode and a second electrode are functionalized so as to bind the protein-of-interest and a third electrode is included whereby the desired pre-determined AC voltage and frequency is applied relative to the first and second electrode to attract or concentrate the protein-of-interest to facilitate binding of the protein-of-interest to the first and second electrode.

In some embodiments, a peptide or polypeptide that is capable of enzymatic recognition and modification can be incorporated at two widely separated points on the enzyme, each chosen so as not to interfere with the function of the enzyme. As is disclosed in more detail in PCT Application No. PCT/US2019/032707, which is incorporated herein by reference in its entirety and for all purposes, protein bioelectronic circuits in which the protein-of-interest is an enzyme can be connected so that the electric current through the enzyme reports on functional motions of the protein. In such circuits, it is generally simplest to interpret signals if no more than one protein-of-interest (or a fragment thereof) is incorporated between each pair of electrodes. In some embodiments, as disclosed in more detail in U.S. Pat. No. 10,379,102, which is incorporated herein by reference in its entirety and for all purposes, methods for trapping and connecting a protein-of-interest (or a fragment thereof) in a bioelectronic circuit can include a first electrode and a second electrode, with each electrode being functionalized with molecules that recognize and provide connections to the protein molecule. In order to follow the motions of a particular enzyme, it is desirable that each electrode pair connect to one, and not more than one enzyme.

However, random functionalization of the electrode gaps of these bioelectronic circuits with enzymes such that the mean occupation number is one results in only about 30% of the electrode pairs having the desired functionalization (a consequence of Poisson statistics). Additionally, devices for detecting the presence of a protein-of-interest are limited to lower levels of concentration (e.g., on the order of nM concentrations) because of the requirement that a molecule diffuse into a target area the size of the gap area (e.g., approximately 10 nm by 10 nm) and do so in a reasonable time (e.g., minutes). Therefore, embodiments of the present disclosure include devices, systems, and methods for positioning no more than one protein-of-interest in a target region between a pair of electrodes, as well as devices, systems, and methods for selectively attracting and increasing the concentration of analytes that may interact with the protein-of-interest.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As noted herein, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, compositions, systems and apparatuses/devices which may further include any and all elements from any other disclosed methods, compositions, systems, and devices, including any and all elements corresponding to detecting one or more target molecules (e.g., DNA, proteins, and/or components thereof). In other words, elements from one or another disclosed embodiments may be interchangeable with elements from other disclosed embodiments. Moreover, some further embodiments may be realized by combining one and/or another feature disclosed herein with methods, compositions, systems and devices, and one or more features thereof, disclosed in materials incorporated by reference. In addition, one or more features/elements of disclosed embodiments may be removed and still result in patentable subject matter (and thus, resulting in yet more embodiments of the subject disclosure). Furthermore, some embodiments correspond to methods, compositions, systems, and devices which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

Also, while some of the embodiments disclosed are directed to detection of a protein molecule, within the scope of some of the embodiments of the disclosure is the ability to detect other types of molecules.

When describing the molecular detecting methods, systems and devices, terms such as linked, bound, connect, attach, interact, and so forth should be understood as referring to linkages that result in the joining of the elements being referred to, whether such joining is permanent or potentially reversible. These terms should not be read as requiring a specific bond type except as expressly stated.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “dielectrophoresis” or “DEP” generally refers to the movement of a polarizable particle when it is subjected to a nonuniform electric field due to the interaction of a polarized particle and spatial gradient of the electric field. Since biological molecules have diverse dielectric properties, DEP can be used to manipulate, transport, separate, and sort different types of biological molecules.

As used herein, “electric field gradient” generally refers to a directional rate of change in an electric field due to the distribution of charges with respect to a particular reference point. An electric field gradient can be generated by a variety of means, including but not limited to, creating a non-uniform alternating electric field between an electrode pair.

### 2. Protein Bioelectronic Devices and Methods

Embodiments of the present disclosure include devices, systems, and methods for manipulating a protein-of-interest into a target position within two electrodes in order to generate a functional bioelectronic circuit.

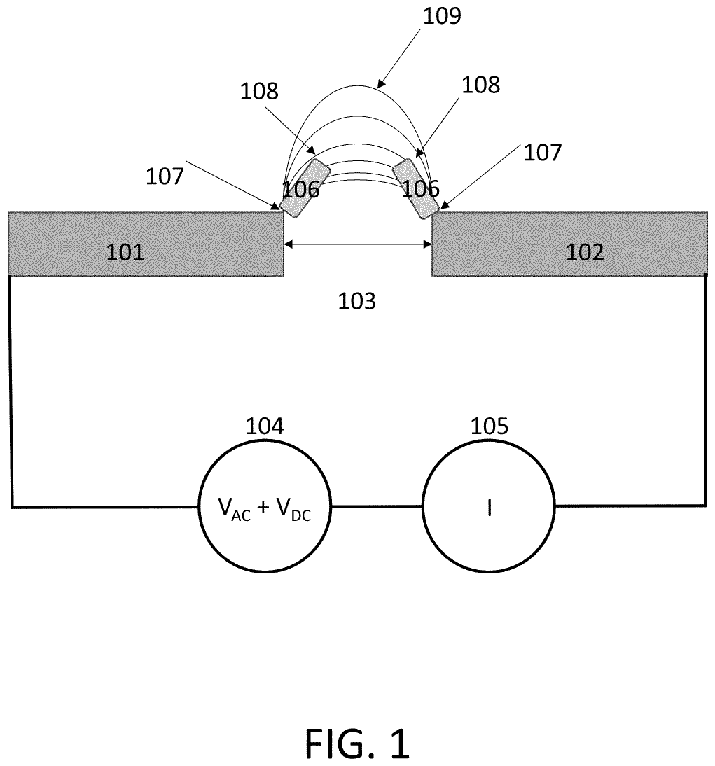

Referring to FIG. 1, a device for positioning a protein-of-interest (e.g., “trapping”) between two electrodes is shown. In this exemplary embodiment, a first electrode 101 can be separated by a gap 103 from a second electrode 102. The electrodes can be functionalized with recognition molecules 106 in which a first end 107 binds specifically to the metal electrodes (e.g., by a thiol-metal bond), while a second end 108 binds specifically to a site on the protein-of-interest. An electric field gradient 109 can be generated by applying an AC voltage (V_{AC}) and a DC voltage (V_{DC}) 104 across the electrode pair. The device of FIG. 1 is also configured for recording the current (I) 105 passing through them. In some embodiments, the DC voltage (V_{DC}) can be zero volts.

In accordance with these embodiments, the gap between the electrodes size can be from about 1 to about 10 nm. In some embodiments, the gap between the electrodes size can be from about 1 to about 8 nm. In some embodiments, the gap between the electrodes size can be from about 1 to about 6 nm. In some embodiments, the gap between the electrodes size can be from about 1 to about 5 nm. In some embodiments, the gap between the electrodes size can be from about 1 to about 4 nm. In some embodiments, the gap between the electrodes size can be from about 2 to about 10 nm. In some embodiments, the gap between the electrodes size can be from about 4 to about 10 nm. In some embodiments, the gap between the electrodes size can be from about 5 to about 10 nm. In some embodiments, the gap between the electrodes size can be from about 6 to about 10 nm.

In some embodiments, the DC voltage is from about 5 mV to about 500 mV. In some embodiments, the DC voltage is from about 50 mV to about 500 mV. In some embodiments, the DC voltage is from about 100 mV to about 500 mV. In some embodiments, the DC voltage is from about 200 mV to about 500 mV. In some embodiments, the DC voltage is from about 300 mV to about 500 mV. In some embodiments, the DC voltage is from about 400 mV to about 500 mV. In some embodiments, the DC voltage is from about 10 mV to about 400 mV. In some embodiments, the DC voltage is from about 5 mV to about 300 mV. In some embodiments, the DC voltage is from about 5 mV to about 200 mV. In some embodiments, the DC voltage is from about 5 mV to about 100 mV.

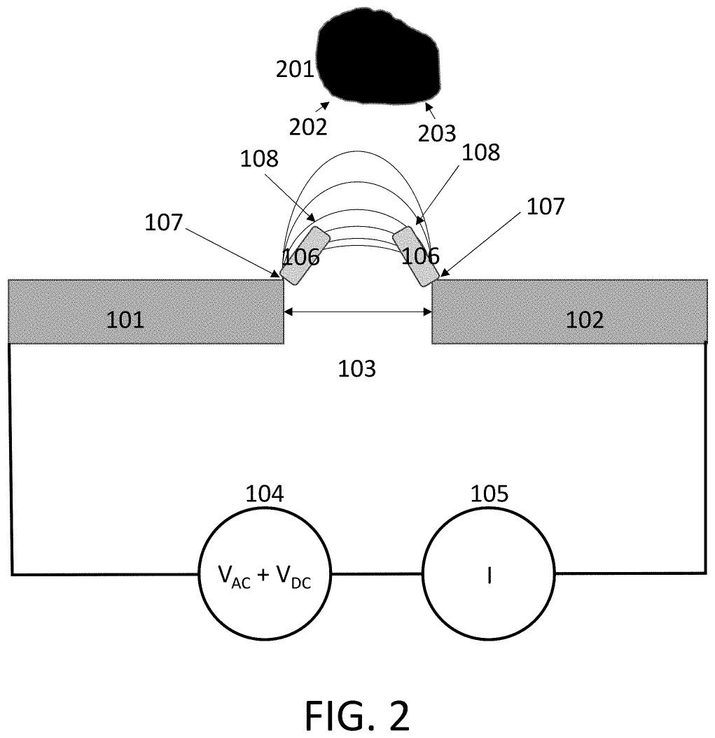

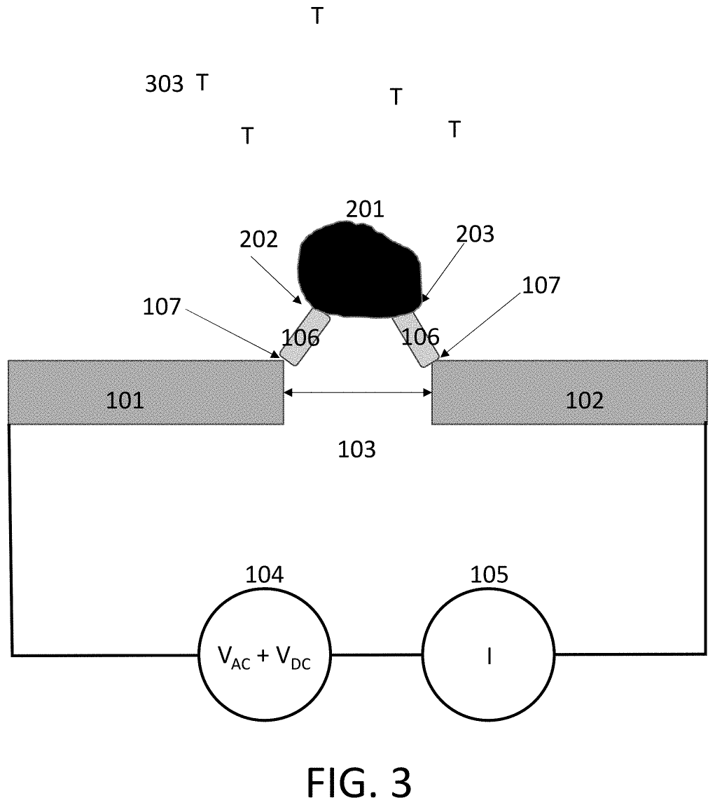

In some embodiments, as shown in FIG. 3, the device can be exposed to a solution of proteins-of-interest 201 comprising two specific binding sites 202, 203 that attach to the sites 108 (see, FIG. 2) on the recognition molecules 106. An AC voltage with a previously set frequency can then be applied to attract the protein 201 into the gap by means of dielectrophoresis. The optimal frequencies and voltage can be determined as described further below. Typical frequencies can be from about 1 kHz and 50 MHz, with some frequencies being between 100 kHz and 5 MHz. In some embodiments, frequencies range from about 1 kHz to about 5 mHz. In some embodiments, frequencies range from about 1 kHz to about 1 mHz. In some embodiments, frequencies range from about 1 kHz to about 500 kHz. In some embodiments, frequencies range from about 1 kHz to about 250 kHz. In some embodiments, frequencies range from about 1 kHz to about 100 kHz. In some embodiments, frequencies range from about 1 kHz to about 50 kHz. In some embodiments, frequencies range from about 100 kHz to about 5 MHz. In some embodiments, frequencies range from about 250 kHz to about 5 MHz. In some embodiments, frequencies range from about 500 kHz to about 5 MHz. In some embodiments, frequencies range from about 1 MHz to about 5 MHz. In some embodiments, frequencies range from about 3 MHz to about 5 MHz, frequencies range from about 5 MHz to about 20 MHz, or frequencies range from about 20 MHz to about 50 MHz.

As described above, an AC voltage can be applied to attract the protein 201 into the gap by means of dielectrophoresis. In some embodiments, the AC voltage is from about 10 mV to about 5 V. In some embodiments, the AC voltage is from about 100 mV to about 5 V. In some embodiments, the AC voltage is from about 250 mV to about 5 V. In some embodiments, the AC voltage is from about 500 mV to about 5 V. In some embodiments, the AC voltage is from about 750 mV to about 5 V. In some embodiments, the AC voltage is from about 1 V to about 5 V. In some embodiments, the AC voltage is from about 2 V to about 5 V. In some embodiments, the AC voltage is from about 3 V to about 5 V. In some embodiments, the AC voltage is from about 4 V to about 5 V. In some embodiments, the AC voltage is from about 10 mV to about 4 V. In some embodiments, the AC voltage is from about 10 mV to about 2 V. In some embodiments, the AC voltage is from about 10 mV to about 1 V. In some embodiments, the AC voltage is from about 10 mV to about 500 mV. In some embodiments, the AC voltage is from about 10 mV to about 250 mV. In some embodiments, the AC voltage is from about 10 mV to about 100 mV. In some embodiments, the AC voltage is from about 50 mV to about 2 V. In some embodiments, the AC voltage is from about 100 mV to about 1 V. In some embodiments, the AC voltage is from about 500 mV to about 1 V.

In some embodiments, binding of a protein-of-interest is indicated by a sudden increase in current (I) 105 at which point the AC voltage is either (1) set to 0 to stop attraction of the protein-of-interest 201 or (2) set to a frequency that repels further capture of the protein 201, thus allowing for predictable single-molecule functionalization of the gap and circumventing the Poisson limit for a randomly functionalized gap.

AC voltages and frequencies for attracting or immobilizing a given protein-of-interest in a solution of a given permittivity and conductivity can be determined (or pre-determined) in various ways. In some embodiments, AC voltages and frequencies for attracting a given protein-of-interest in a particular solvent can be determined by fluorescently labelling a given protein-of-interest and sweeping both the voltage and AC frequency while measuring the concentration rate optically. In other embodiments, AC voltages and frequencies for attracting a given protein-of-interest can be determined by sweeping both the voltage and AC frequency while measuring the capacitance on an integrated capacitive sensor. In still other embodiments, AC voltages and frequencies for attracting a given protein-of-interest can be determined by sweeping both the voltage and AC frequency while measuring the impedance between the nanogaps. AC voltages and frequencies for repelling a given protein-of-interest in a solution of a given permittivity and conductivity can be determined (or pre-determined) in various ways. In some embodiments, AC voltages and frequencies for repelling a given protein-of-interest can be determined by fluorescently labelling a given protein-of-interest and sweeping both the voltage and AC frequency and observing movement away from the nanogap. To aid in observation of repelling a given protein-of-interest, it is generally beneficial to first attract a number of given proteins-of-interest.

In some embodiments AC voltages and frequencies for attracting or repelling a given protein-of-interest can be determined by dielectric spectroscopy or electrochemical impedance spectroscopy.

As would be recognized by one of ordinary skill in the art based on the present disclosure, between the optimal frequency for attracting and repelling a given protein-of-interest there can exist a cross-over point at which the given protein-of-interest experiences no force. Determining this point can be useful for identifying voltages and frequencies in a solution of a given permittivity and conductivity that permit manipulation of additional analytes (e.g., which may bind the protein-of-interest), without manipulating the bound protein positioned in the electrode gap that forms the bioelectrical circuit.

The positioning of a single protein-of-interest can be indicated by currents (I) 105 that increase abruptly from about 1 pA to about 10 pA, to about 100 pA to about 1000 pA or more. In some embodiments, the trapping of a single protein-of-interest is indicated by an abrupt drop in the measured impedance across the circuit. In some embodiments the binding of the single protein-of-interest causes a characteristic change in the conductance fluctuations.

The positioning of a single molecule of a protein-of-interest can be detected by various methods. In some embodiments, a single molecule of a protein-of-interest can be detected through the application of a low-frequency AC signal and lock-in amplifier. In other embodiments, a single molecule of a protein-of-interest can be detected through the application of a DC offset added to the high frequency AC signal used to attract the desired protein, which allows for the detection of the sudden increase in DC current, as described above.

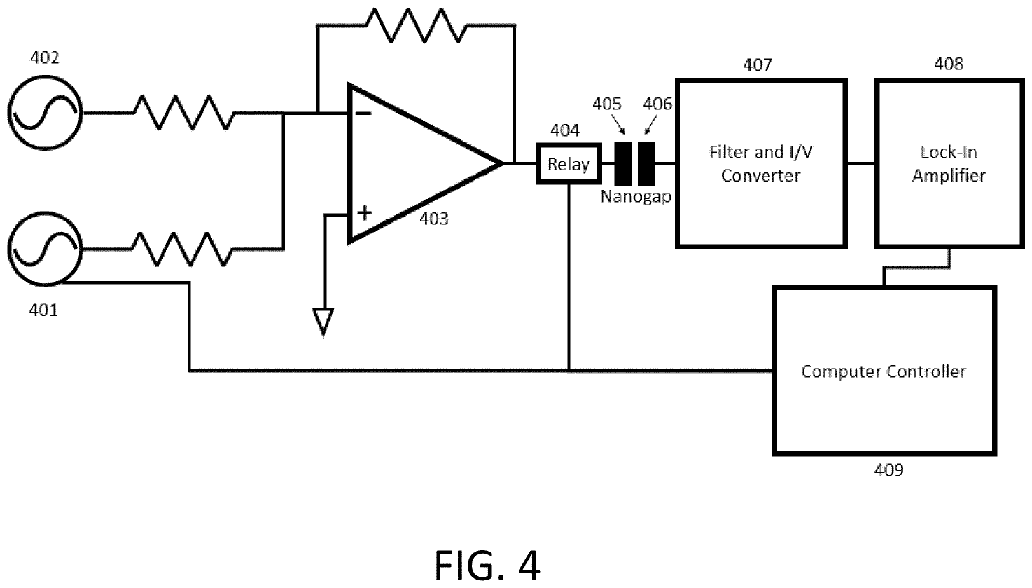

In accordance with these embodiments, as shown in FIG. 4, two AC frequencies 401, 402 can be superimposed via a summing amplifier 403. The first, higher frequency signal 401 can be the optimized frequency for attracting a given protein, while the second frequency 402 is lower, for example, between 0.1 to 10,000 Hz with a peak-to-peak voltage between 10 to 500 mV. The output of the summing amplifier 403 can then be passed through a computer-controlled relay 404 and then on to one of electrodes 405, containing a gap between them to bind the desired protein. The other electrode 406 can then be connected to a transimpedance amplifier with a low-pass filter 407 to remove the high frequency optimized for attracting a given protein. The output of this can then be connected to a lock-in amplifier 408 and computer controller 409 to detect the abrupt change in the low frequency current passing through the protein-of-interest once captured and break the circuit via the relay 404 to stop attracting additional proteins. In one embodiment, upon detection of binding of the desired protein-of-interest, the computer controller changes frequency 401 so as to repel an additional proteins, but not break the binding of the already bound protein.

It will be recognized by one of ordinary skill in the art based on the present disclosure that these and similar methods can be used for concentrating an analyte molecule that is a substrate (or a potential substrate) for a protein-of-interest (e.g., enzyme) attached so as to span the electrode gap. For example, FIG. 3 shows an enzyme 201 trapped in the gap by the recognition molecules 106. The substrate for the enzyme is shown as “T” 303. If the substrate is present is a low concentration, the time to bind a single target molecule may be excessively long. However, the analyte T can be attracted to the enzyme using the devices, systems, and methods described herein. For example, an alternating electric field can be generated between the two electrodes forming the gap, resulting in an electric field gradient extending out into the solution with the electric field maximum at the edges of the two electrodes. When the desired analyte is subjected to an appropriately polarizing alternating electric field, the analyte experiences a force in the direction of the electric field gradient until it reaches the electric field maximum at the electrode edges. The analyte can similarly be repelled by application of an appropriately polarizing electric field causing the analyte to experience a force in the direction of the electric field gradient until it reaches the electric field minimum or another force dominates. In one embodiment, the AC frequency applied is chosen so as to minimize the force exerted on the protein attached to the electrode gap. Means of polarizing an analyte include, but are not limited to, counterion relaxation, interfacial polarization, dielectric dispersion, dipole relaxation, and the like.

In accordance with these embodiments, the devices and methods of the present disclosure can be used to generate a bioelectronic circuit with any protein-of-interest and any corresponding target analyte. For example, proteins-of-interest can include, but are not limited to, enzymes, cell surface receptors, transmembrane proteins, antibodies, intracellular signaling proteins, nucleic acid binding proteins, secretory proteins, and the like, including any engineered proteins or polypeptides (e.g., fusion proteins, chimeric proteins, recombinant proteins, and the like) and corresponding fragments, derivatives and variants thereof. In some embodiments, the protein-of-interest is an enzyme, including but not limited to, a polymerase, a nuclease, a proteasome, a glycopeptidase, a glycosidase, a kinase and an endonuclease. In some embodiments, proteins-of-interest and target analytes include, but are not limited to, receptor proteins, where the target molecules are hormones, neurotransmitters, growth factors, toxins, small molecule pharmaceuticals, and the like. In some embodiments, target proteins include, but are not limited to, engineered Major Histocompatibility Complex (MHC) proteins where the target molecules are peptides, pathogen antigens where the target molecules are biologics (e.g., engineered therapeutic biologics, antibodies, or fragments thereof), including monoclonal, neutralizing, and synthetic antibodies, and engineered CRISPR associated protein where the target is DNA or RNA.

In some embodiments, the target analyte can include, but is not limited to, a biopolymer and/or a subunit of a biopolymer. In some embodiments, the analyte is capable of interacting with the protein-of-interest in the bioelectronic devices described herein. In some embodiments, the analyte is a biopolymer or subunit of a biopolymer selected from the group consisting of a DNA molecule, an RNA molecule, a peptide, a polypeptide, and a glycan. In some embodiments, the methods of the present disclosure include generating a bioelectronic circuit as part of the devices and systems described herein to sequence a biopolymer. In some embodiments, the present disclosure includes methods for sequencing a polynucleotide using a bioelectronic device that obtains a bioelectronic signature of polymerase activity based on current fluctuations as complementary nucleotidepolyphosphate monomers are incorporated into a template polynucleotide.

In accordance with these embodiments, the devices and methods of the present disclosure can include any type of recognition molecules. For example, the recognition molecules can be antibodies (or any antigen binding fragments thereof) and the protein-of-interest can be a corresponding antigen. In some embodiments, the recognition molecules can be a receptor protein, and the protein-of-interest can be a corresponding ligand. In some embodiments, the recognition molecules can be engineered to include a chemical or polypeptide moiety that binds to a corresponding protein-of-interest and/or a corresponding acceptor moiety linked to the protein-of-interest. In some embodiments, the protein-of-interest can be engineered to include a chemical or polypeptide moiety that binds to a corresponding recognition molecule and/or a corresponding acceptor moiety linked to the recognition molecule.

In some embodiments, it may be desirable to trap additional proteins in the same gaps, with the desired number being from about 2 to about 100. It will be recognized by one of ordinary skill in the art based on the present disclosure that the methods disclosed herein to attract and bind a specific protein-of-interest could also be applied serially to trap and bind additional target proteins. To identify the binding of additional proteins, a step change in current through circuit 105 or a step change in impedance may be used. Additionally, the methods disclosed herein to attract or repel a specific protein-of-interest can be used to selectively functionalize a large number of gaps with different proteins for multiplexed detection or to leave electrodes unfunctionalized for future use.

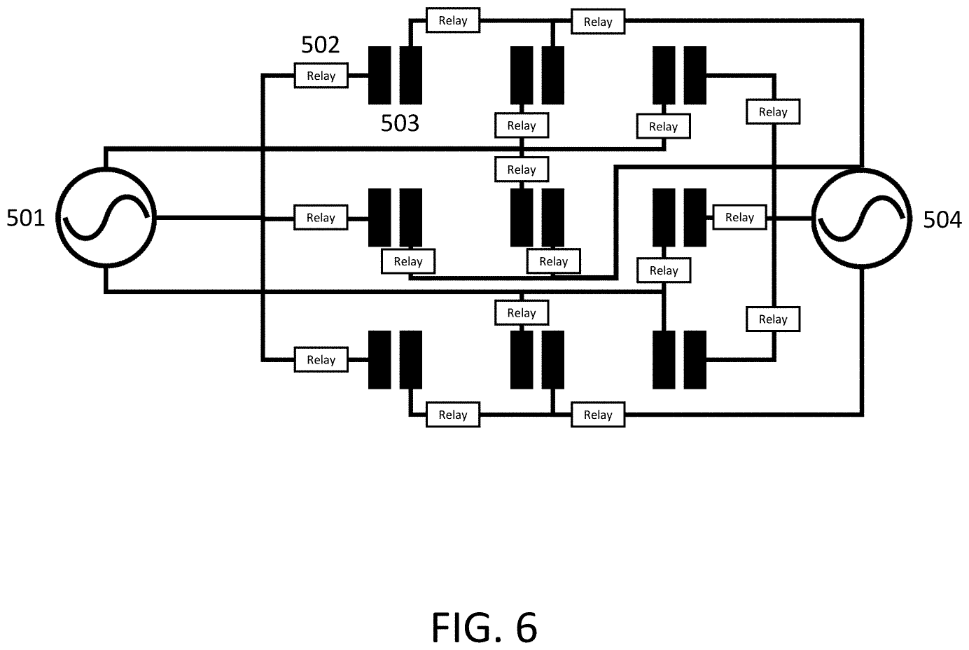

In some embodiments, as shown in FIG. 6, gaps 503 desired to be functionalized by a given protein-of-interest have an AC voltage and frequency 501 applied to them through an activated relay 502, chosen so as to attract the protein while gaps not desired to be functionalized have an AC voltage and frequency 504 applied to them through an activated relay, chosen so as to repel the protein. As one of ordinary skill in the art would recognize based on the present disclosure, this process may be repeated for subsequent different proteins-of-interest to functionalize additional gaps.

In some embodiments, when an undesired protein or proteins functionalizes a gap, an AC frequency and voltage may be applied to the gap to remove the protein by applying a force sufficient to break the binding. An undesired protein may be identified by characteristic signals measured through it upon binding the functionalized gaps. An undesired protein may be determined by the by currents (I) 105 passed through it upon binding.

In some embodiments, when an undesired analyte binds or is recognized by the protein functionalized in the gap, an AC frequency and voltage may be applied to the gap to remove the analyte by applying a force sufficient to break the specific binding. An undesired analyte may be identified by characteristic signals measured upon binding or being recognized by the protein functionalized in the gap. An undesired analyte may be determined by the by currents (I) 105 passed through it upon binding.

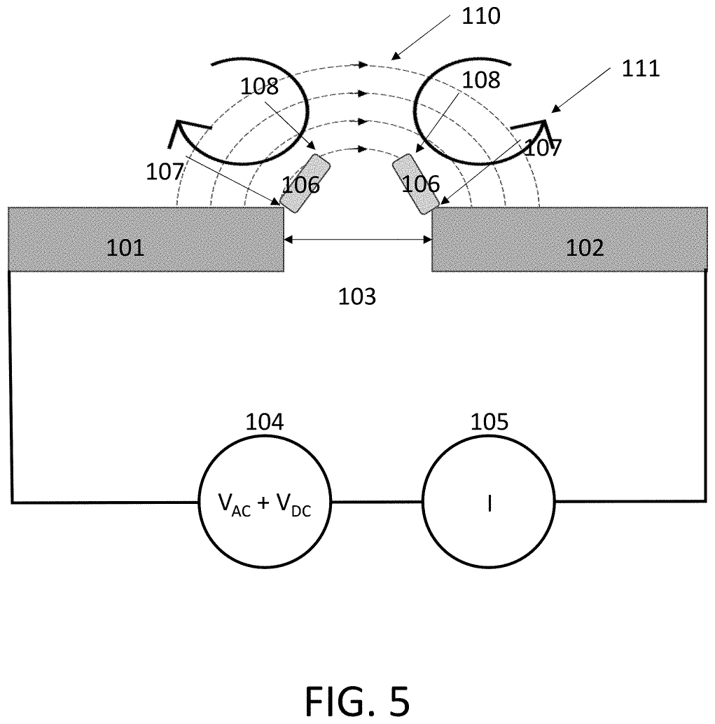

Certain AC electric fields, in the presence of a conductive solution, can lead to localized Joule heating of the solution leading to electrothermal flows, as shown in FIG. 5 (111). In some embodiments, electrothermal flows, which move solution through convective flows, can be beneficial in drawing analytes towards the gap between an electrode pair for subsequent sensing via the bioelectronic circuit. In another embodiment, an AC electric field frequency is chosen so as to minimize electrothermal flows, but increase the temperature locally near the gap between an electrode pair through Joule heating, enhancing diffusion, which can be beneficial for temperature dependent dynamics. In some embodiments, the force of the fluid generated by the electrothermal flow may be used to remove an attached protein to the gap to permit functionalization again or to leave the gap unfunctionalized. At yet other AC electric field frequencies, the application of the electric field can induce electro-osmotic flow at the edges of the exposed electrodes. Electro-osmotic flow can be used to convect proteins-of-interest to the gap for functionalization and analytes-of-interest to the attached protein for sensing. In yet other embodiments, the gaps for the aforementioned attraction and repelling of proteins and analytes-of-interest are separate from the gaps used for sensing and are within a distance from about 0.1 to about 10 μm. AC electric fields may be applied to these devices to manipulate proteins and analytes to attract, repel, convect, or increase the diffusion rate of the desired proteins and analytes to the gap.

Embodiments of the present disclosure also include the use of a carrier or carriers to generate the bioelectronic devices of the present disclosure. In accordance with these embodiments, a carrier can be any agent or particle that can be polarized, such that when coupled to a protein-of-interest, the carrier facilitates the formation of a bioelectronic circuit (as described above) when exposed to an electric field gradient. Exposing the carrier and protein-of-interest in solution to a suitable electric field gradient results in dielectrophoresis, thereby attracting the protein-of-interest to the proper position between an electrode pair or within the vicinity of an electrode pair to be functionalized. Carriers permit the formation of bioelectronic circuits even when the protein-of-interest is not easily polarizable and subjected to dielectrophoresis.

In some embodiments the carrier or carriers are elements of the bioelectronic circuit and conductive and are chosen so as to optimize the operation of a bioelectronic circuit. In other embodiments the carrier or carriers are not components of the bioelectronic circuit. In yet other embodiments the carrier are chosen with a desired polarizability such that specific carriers may be attracted to specific electrode pairs, permitting the selective formation of a number of unique bioelectronic circuits from the same solution. Carriers may also be chosen to have a polarizability within a given solution that optimizes the application of dielectrophoresis and the formation of bioelectronic circuits.

### 3. Systems and Methods

Embodiments of the present disclosure also include a system for direct electrical measurement of protein activity. In accordance with these embodiments, the system includes any of the devices described herein, a means for introducing a chemical entity that is capable of interacting with the protein-of-interest, a means for applying a voltage bias between the first and second electrodes that is 100 mV or less, and a means for monitoring fluctuations that occur as the chemical entity interacts with the protein-of-interest.

Embodiments of the present disclosure also include an array comprising a plurality of any of the bioelectronic devices described herein. In some embodiments, the array includes a means for introducing an analyte capable of interacting with the protein, a means for applying a voltage bias between the first and second electrodes that is 100 mV or less, and a means for monitoring fluctuations that occur as the chemical entity interacts with the protein. The array can be configured in a variety of ways, as would be appreciated by one of ordinary skill in the art based on the present disclosure. In some embodiments, the array comprises a plurality of bioelectronic devices comprising a plurality of proteins-of-interest. In some embodiments, a plurality of pre-determined AC voltages and frequencies corresponding to the plurality of proteins-of-interest are used with a plurality of electrode pairs (e.g., depending on what is being measured by the bioelectronic devices). In accordance with these embodiments, the present disclosure also includes methods for using the arrays, comprising exposing a plurality of electrode pairs to a solution(s) comprising a plurality of proteins-of-interest, and applying pre-determined AC voltage(s) and frequency(ies) corresponding to the plurality of proteins-of-interest to the plurality of electrode pairs.

Embodiments of the present disclosure also include methods of measuring electronic conductance through a protein-of-interest using any of the devices and systems described herein. In accordance with these embodiments, the present disclosure includes methods for direct electrical measurement of protein activity. In some embodiments, the method includes introducing an analyte capable of interacting with the protein to any of the bioelectronic devices described herein, applying a voltage bias between the first and second electrodes that is 100 mV or less, and observing fluctuations in current between the first and second electrodes that occur when the analyte interacts with the protein. In some embodiments, the analyte is a biopolymer selected from the group consisting of a DNA molecule, an RNA molecule, a peptide, a polypeptide, and a glycan. In some embodiments, methods of the present disclosure include use of the devices and systems described herein to sequence a biopolymer. In some embodiments, the present disclosure includes methods for sequencing a polynucleotide using a bioelectronic device that obtains a bioelectronic signature of polymerase activity based on current fluctuations as complementary nucleotidepolyphosphate monomers are incorporated into the template polynucleotide.

As described further herein, the devices, systems, and methods of the present disclosure can be used to generate a bioelectronic signature of an enzyme-of-interest, which can be used to determine the sequence of any biopolymer (e.g., polynucleotide). In some embodiments, the enzyme-of-interest can be a polymerase, and various aspects of a bioelectronic signature of a polymerase as it adds nucleotide monomers to a template polynucleotide strand can be used to determine the sequence of that template polynucleotide. For example, a bioelectronic signature of polymerase activity can be based on current fluctuations as each complementary nucleotide monomer is incorporated into the template polynucleotide. In some embodiments, the bioelectronic device used to generate a bioelectronic signature comprises a polymerase functionally coupled to both a first electrode and a second electrode using the adaptor polypeptides of the present disclosure. The term “nucleotide” generally refers to a base-sugar-phosphate combination and includes ribonucleoside triphosphates ATP, UTP, CTG, GTP and deoxyribonucleoside triphosphates such as dATP, dCTP, dITP, dUTP, dGTP, dTTP, or derivatives thereof.

As one of ordinary skill in the art will readily recognize and appreciate after having benefited from the teachings of the present disclosure, the methods described herein can be used with any bioelectronic device that senses the duration of the open and closed states of an enzyme (e.g., polymerase). Exemplary devices include, but are not limited to, the bioelectronic devices and systems disclosed in U.S. Pat. No. 10,422,787 and PCT Appln. No. PCT/US2019/032707, both of which are herein incorporated by reference in their entirety and for all purposes. Additionally, it will be readily recognized and appreciated by those of ordinary skill in the art based on the present disclosure that the forgoing embodiments apply equally to (and include) sequencing RNAs with the substitution of rNTPs for dNTPs and the use of an RNA polymerase.

Further, one of ordinary skill in the art would readily recognize and appreciate that the methods described herein can be used in conjunction with other methods involving the sequencing of a biopolymer. In particular, the various embodiments disclosed in PCT Application No. PCT/US21/19428, which is herein incorporated by reference in its entirety, describes the interpretation of current fluctuations generated by a DNA polymerase as it actively extends a template, and how signal features (e.g., bioelectronic signature) may be interpreted in terms of the nucleotide being incorporated, and thus, how these signals can read the sequence of the template. This approach utilizes features of the signal that vary in time. For example, the time that the polymerase stays in a low current state reflects the concentration of the nucleotidetriphosphate in solution. If the concentration of a particular nucleotide triphosphate is low, then the polymerase must stay open for a longer time in order to capture the correct nucleotide, and since the open conformation of the polymerase corresponds to a lower current, the dip in current associated with the open state lasts for longer. Additionally, the various embodiments disclosed in PCT Application No. PCT/US20/38740, which is herein incorporated by reference in its entirety, describes how the base-stacking polymerization rate constant differences are reflected in the closed-state (high current states) so that the duration of these states may also be used as an indication of which one of the four nucleotides is being incorporated. It can be desirable to be able to use the amplitude of the signal as yet an additional contribution to determining sequence. Further, the various embodiments disclosed in PCT Application No. PCT/US21/17583, which is herein incorporated by reference in its entirety, describes methods that utilize a defined electrical potential to maximize electrical conductance of a protein-of-interest (e.g., polymerase), which can serve as a basis for the fabrication of enhanced bioelectronic devices for the direct measurement of protein activity. Additionally, the various embodiments disclosed in PCT Application No. PCT/US21/30239, which is herein incorporated by reference in its entirety, describes methods for sequencing a polynucleotide using a bioelectronic device that obtains a bioelectronic signature of polymerase activity based on current fluctuations as complementary nucleotidepolyphosphate monomers having distinctive charges are incorporated into the template polynucleotide.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

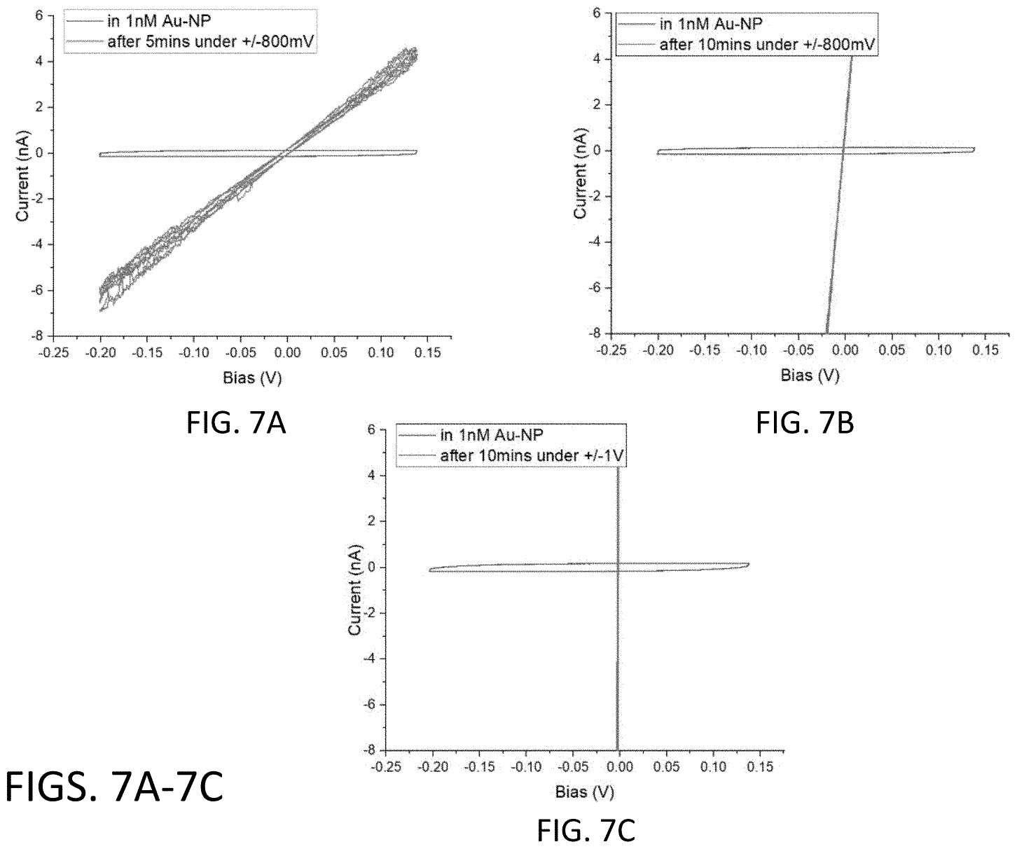

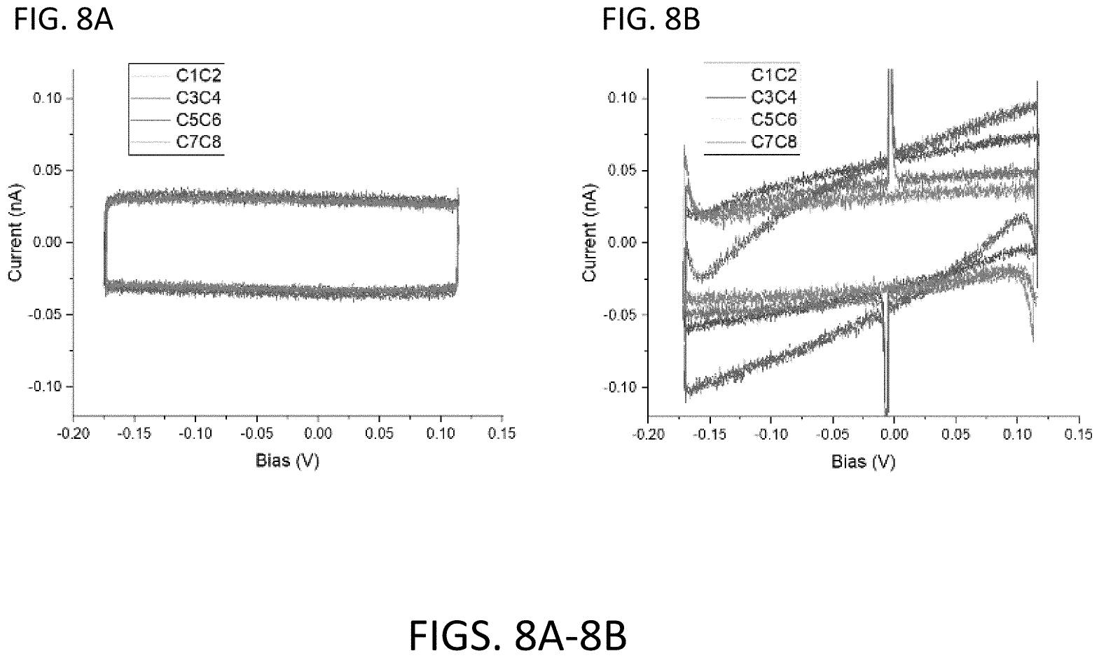

As shown in FIGS. 7 and 8, experiments were conducted to assemble the circuits of the present disclosure using a carrier. In this example, experiments were conducted using gold nanoparticle carriers (AuNPs) approximately 20 nm diameter that were functionalized with carboxylic acid. AuNPs having 5 nm diameters were also tested, and produced essentially the same results (data not shown but can be provided up on request). The AuNPs were exposed to an electrode pair at a concentration of about 1 nM in double-distilled deionized water using an applied square wave at 2 MHz and a potential of 1-2 volts peak-to-peak.

The exemplary data in FIGS. 7A-7C includes three separate bioelectronic devices demonstrating increased conductance readings (red trace) after DEP in 1 nM AuNPs after 5 mins of an applied square wave of +/−800 mV amplitude (FIG. 7A), in 1 nM AuNPs after 10 mins of an applied square wave of +/−800 mV amplitude (FIG. 7B), and in 1 nM AuNPs after 5 mins of an applied square wave of +/−1V amplitude (FIG. 7C). The gray trace is showing no conductance before DEP, despite the present of the AuNPs. The data in FIGS. 8A-8B include controls showing no conductance before the addition of water and AuNPs (FIG. 8A) and in water before addition of AuNPs (FIG. 8B).

Taken together, these data indicated that both longer duration and increased DEP force resulted in an increase in conductance after DEP, demonstrating the ability to tune the number of carriers drawn into a given junction. These data provide proof-of-principle for generating DEP with devices designed for bioelectronics, and that a carrier (e.g., AuNPs and other easily polarized carriers) can be used to assemble the bioelectronic circuits of the present disclosure.

What is claimed is:

1. A method for generating a bioelectronic circuit, the method comprising:
generating an electric field gradient between an electrode pair functionalized with recognition molecules;
exposing the electrode pair to a solution comprising a plurality of proteins-of-interest, wherein each of the plurality of proteins-of-interest comprises two binding sites for interacting with the recognition molecules on the electrode pair; and
applying a pre-determined AC voltage and frequency to the electrode pair and attracting a protein-of-interest to the recognition molecules on the electrode pair;
wherein binding of a single protein-of-interest to the electrode pair generates a functional bioelectronic circuit.

2. The method of claim 1, wherein the electric field gradient is generated by applying an initial AC voltage and an initial DC voltage across an electrode pair.

3. The method of claim 1, wherein the pre-determined AC voltage and frequency applied result in dielectrophoresis, thereby attracting the single protein-of-interest to the electrode pair and facilitating binding of the recognition molecules to the binding sites of the protein-of-interest.

4. The method of claim 1, wherein the binding of the single protein-of-interest causes an increase in current from about 1-10 pA to about 100-1000 pA across the circuit.

5. The method of claim 1, wherein the binding of the single protein-of-interest causes a decrease in impedance across the circuit.

6. The method of claim 1, wherein the method further comprises reducing the pre-determined AC voltage applied to the electrode pair upon the increase in current or decrease in impedance.

7. The method of claim 6, wherein reducing the pre-determined AC voltage applied to the electrode pair upon the increase in current or decrease in impedance stops the attraction of a second protein-of-interest to the electrode pair.

8. The method of claim 1, wherein the method further comprises adjusting the pre-determined AC frequency applied to the electrode pair upon the increase in current or decrease in impedance.

9. The method of claim 8, wherein adjusting the pre-determined AC frequency applied to the electrode pair upon the increase in current or decrease in impedance repels a second protein-of-interest from the electrode pair.

10. The method of claim 1, wherein each electrode in the electrode pair is separated by a gap from about 1 nm to about 10 nm.

11. The method of claim 2, wherein the initial DC voltage is from about 5 mV to about 500 mV.

12. The method of claim 1, wherein the pre-determined AC frequency is from about 1 kHz to about 50 MHz.

13. The method of claim 1, wherein the method further comprises exposing the electrode pair to at least a second plurality of proteins-of-interest, and applying a second pre-determined AC voltage and frequency corresponding to the second plurality of proteins-of-interest to the electrode pair.

14. The method of claim 1, wherein the method further comprises exposing a second electrode pair to a second solution comprising a second plurality of proteins-of-interest, and applying a second pre-determined AC voltage and frequency corresponding to the second plurality of proteins-of-interest to the second electrode pair.

15. The method of claim 1, wherein the protein-of-interest is selected from the group consisting of an enzyme, a cell surface receptor, a transmembrane protein, an antibody, an intracellular signaling protein, a growth factor, a nucleic acid binding protein, a secretory protein, viral structural proteins, membrane fusion protein, and any fragments, derivatives, or variants thereof.

16. The method of claim 1, wherein the protein-of-interest is selected from the group consisting of a polymerase, a nuclease, a proteasome, a glycopeptidase, a glycosidase, a kinase and an endonuclease.

17. The method of claim 1, wherein the recognition molecules are selected from the group consisting of antibodies, antigen, receptors, and ligands.

18. The method of claim 1, wherein the protein-of-interest is coupled to a carrier.

19. A method for increasing concentration of an analyte at a bioelectronic circuit, the method comprising:
exposing a bioelectronic circuit to a solution comprising a plurality of analytes, the bioelectronic circuit comprising an electrode pair bound to a protein-of-interest; and
generating an electric field gradient between the electrode pair to polarize the plurality of analytes, thereby forcing the analytes to reach an electric field maximum.

20. The method of claim 19, wherein the electric field gradient is generated by applying an initial AC voltage and an initial DC voltage across an electrode pair.

21. The method of claim 19, wherein the protein-of-interest is selected from the group consisting of an enzyme, a cell surface receptor, a transmembrane protein, an antibody, an intracellular signaling protein, a nucleic acid binding protein, a secretory protein, and any fragments, derivatives, or variants thereof.

22. The method of claim 19, wherein the protein-of-interest is selected from the group consisting of a polymerase, a nuclease, a proteasome, a glycopeptidase, a glycosidase, a kinase and an endonuclease.

23. The method of claim 19, wherein the plurality of analytes is a biopolymer or a subunit of a biopolymer.

24. A method for decreasing concentration of an analyte at a bioelectronic circuit, the method comprising:
exposing a bioelectronic circuit to a solution comprising a plurality of analytes, the bioelectronic circuit comprising an electrode pair bound to a protein-of-interest; and
generating an electric field gradient between the electrode pair to polarize the plurality of analytes, thereby forcing the analytes to reach an electric field minimum.

25. The method of claim 24, wherein the electric field gradient is generated by applying an initial AC voltage and an initial DC voltage across an electrode pair.

26. The method of claim 24, wherein the protein-of-interest is selected from the group consisting of an enzyme, a cell surface receptor, a transmembrane protein, an antibody, an intracellular signaling protein, a nucleic acid binding protein, a secretory protein, and any fragments, derivatives, or variants thereof.

27. The method of claim 24, wherein the plurality of analytes is a biopolymer or a subunit of a biopolymer.
